(19) United States
(12) Patent Application Publication
(10) Pub. No.: US 2004/0029842 A1
(43) Pub. Date: Feb. 12, 2004

(54) Triglyceride lowering, common cold and pneumonia prevention composition comprising tetracycline, and methods of treating or preventing diseases using same

(76) Inventor: Wallace J. Gardner, Cambridge, MA (US)

Correspondence Address: Kevin S. Lemack, Nields & Lemack, Suite 7, 176 E. Main Street, Westboro, MA, 01581, US

(21) Appl. No.: 10/424,270

(22) Filed: Apr. 28, 2003

Related U.S. Application Data
(63) Continuation-in-part of application No. 10/023,017, filed on Dec. 18, 2001, now Pat. No. 6,610,274.

(51) Int. Cl. A61K 31/65

(52) U.S. Cl. 514/152

(57) ABSTRACT

Therapeutic composition having triglyceride blood level lowering activity, total cholesterol blood level lowering activity, LDL blood level lowering activity, HDL blood level raising activity and activity for preventing the common cold and pneumonia. The therapeutic composition is a formulation comprising an antibiotic, preferably a tetracycline, most preferably doxycycline, which optionally has not been chemically modified to eliminate antimicrobial efficacy. The antibiotic is preferably in a liquid vehicle, preferably one that contains at least 20%, most preferably 26% alcohol by volume. The therapeutic composition is preferably in local delivery form and is self-administered orally.

This application is a continuation-in-part of Ser. No. 10/023,017 filed on Dec. 18, 2001, the disclosure of which is hereby incorporated by reference.

### BACKGROUND OF THE INVENTION

The accumulation of bacteria in the oral cavity, such as on the teeth or tongue has been identified as a contributor or cause of various inflammatory conditions, including gingivitis, periodontitis and other gum diseases. Treatment of the oral cavity with antibiotics to reduce or eliminate the effects of microorganisms is known. For example, broad spectrum antibiotics such as tetracyclines and metronidazole have been used in the treatment of periodontal disease to reduce oral cavity microflora. Typically such use has been systemic, which can result in various undesirable side effects, including the threat or danger of building allergies or immunity to the antibiotic, overgrowth of opportunistic yeast and fungi and intestinal disturbances.

Many other common inflammatory diseases, such as sinusitis, diseases of the gastrointestinal tract (including those that manifest themselves in stomach and bowel problems), the common cold, influenza, allergies, halitosis, pneumonia, etc., also may be caused by viruses and/or microorganisms. Often the source of the microorganisms and viruses is the sinuses, typically via the oral cavity, especially the ear, nose and throat passages. Once the microorganisms and/or viruses are resident in the oral cavities or sinuses (e.g., the maxillary, frontal, ethmoid and ophenoidal), they can continually cause inflammation and infection through circulation in the blood stream. Continual reduction or elimination of these microorganisms and viruses would reduce chronic infection in the body.

Elevated blood levels of triglycerides, total cholesterol and LDL, as well as reduced blood levels of HDL, have been linked to heart disease. Many pharmaceuticals currently on the market to lower these elevated levels have adverse side effects, including liver damage.

It therefore would be desirable to provide a therapeutic composition and kit that reduces elevated blood levels of triglycerides, total cholesterol and LDL, raises blood levels of HDL, and that does not suffer from the adverse side effects of existing medications.

It further would be desirable to provide a therapeutic composition and kit that effectively prevents the common cold and pneumonia.

It is still a further object of the present invention to enhance the effect of systemic antibiotics.

### SUMMARY OF THE INVENTION

The problems of the prior art have been overcome by the present invention, which provides a therapeutic composition and kit having anti-infective activity, tryglyceride blood level lowering activity, total cholesterol blood level lowering activity, LDL blood level lowering activity, and/or HDL blood level raising activity, as well as methods of treating various diseases or conditions by administering the compositions. In a preferred embodiment, the therapeutic composition is a formulation comprising an antibiotic, preferably a tetracycline, most preferably doxycycline, which has not been chemically modified to eliminate antimicrobial efficacy. The antibiotic is preferably in a liquid vehicle, preferably one that contains at least 20%, most preferably 26%, alcohol by volume. The therapeutic composition is preferably in local delivery form and is preferably self-administered orally or via the nasal cavity. The therapeutic composition most preferably is a self-delivered formulation in local delivery form that consists essentially of a tetracycline, most preferably doxycycline, which has not been chemically modified to eliminate antimicrobial efficacy, and a liquid vehicle, more preferably one which contains at least 20%, preferably at least 26% alcohol by volume, and most preferably one which consists essentially of sterile water or a rinse as defined below or the like, which tetracycline is preferably present in the formulation in the amount of between 50 to 100 mgs per ounce of liquid vehicle.

Administration of the therapeutic composition of the present invention treats diseases that originate from the sinuses, usually via the oral and/or nasal cavities, and are initiated by contaminants, such as viruses or microorganisms, in the sinuses entering the bloodstream including but not limited to periodontal disease, sinusitis, gingivitis, the common cold, sore throat, influenza, allergies (particularly to tree pollen), resistant pneumonia, diseases of the gastrointestinal tract, inflammatory diseases such as rheumatoid arthritis, cancer, ulcers, heart disease, high cholesterol, high triglyceride levels, high LDL levels, low HDL levels, etc.

In one embodiment of the present invention, the therapeutic compositions of the present invention have a triglyceride and total cholesterol lowering activity, as well as an HDL raising activity. Compositions useful for this purpose are a formulation comprising an antibiotic, preferably a tetracycline, most preferably doxycycline, in a liquid vehicle, the liquid vehicle preferably containing at least 20%, preferably at least 26% alcohol by volume. The tetracycline is optionally one that has not been chemically modified to eliminate antimicrobial activity. The therapeutic composition of this embodiment is preferably in local delivery form, and is preferably self-administered orally.

A further embodiment of the present invention relates to a pharmaceutical preparation that includes a kit comprising the therapeutic compositions mentioned above along with instructions for use in treating various maladies, including elevated blood levels of triglycerides, elevated blood levels of cholesterol, increasing HDL blood levels, treating periodontal disease, and/or preventing the common cold and pneumonia.

The compositions of the present invention also have a beneficial effect of enhancing systemic antibiotics.

### DETAILED DESCRIPTION OF THE INVENTION

The therapeutic compositions of the present invention are local delivery compositions that have anti-infective activity, a triglyceride blood level lowering activity, total cholesterol blood level lowering activity, LDL blood level lowering activity, an HDL blood level raising activity, pneumonia and/or cold prevention activity, and activity for enhancing the effect of systemic antibiotics, and comprise an antibiotic, preferably a tetracycline which optionally has not been chemically modified to eliminate antimicrobial efficacy. The compositions are effective to prevent pollutants from passing from oral passages into the blood stream.

The tetracyclines useful in the compositions of the present invention include apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin, and tetracycline. Doxycycline is particularly preferred.

The therapeutic composition of the invention also includes a carrier that is any compatible, nontoxic substance suitable to deliver the antibiotic. Carriers can include sterile water, sodium chloride (e.g. 0.9%), alcohol, fats, waxes, inert solids and even liposomes. Preferably, the antibiotic is in a liquid vehicle. Water is a preferred liquid for the liquid vehicle and the vehicle even more preferably comprises at least about 20% alcohol by volume, more preferably 20-40% alcohol and most preferably about 20-35% alcohol. Preferably, the therapeutic composition does not include a morpholinoamino alcohol or pharmaceutically-acceptable salt thereof. One particularly preferred formulation includes a mixture of doxycycline with 0.064% thymol, 0.092% eucalyptol, 0.060% methylsalicylate, 0.042% menthol, 26.9% alcohol, poloxamer 407, water, sodium benzoate and caramel. Other suitable formulations include a mixture of doxycycline with commercially available mouthwashes, oral rinses or anti-plaque formulations. Pharmaceutically acceptable adjuvants may also be incorporated into the therapeutic composition. In certain therapeutic compositions of the invention, antiphlogisitic agents are not preferred. The compositions of the present invention also may contain various inactive ingredients, such as sweeteners (natural and artificial), flavorings (natural or artificial), colorants, preservatives, etc.

The amount of antibiotic used in the formulation depends upon the frequency of the dose, the particular infection or malady and the severity of the infection or malady being treated or prevented, the method of local administration of the composition and the effect desired (including whether the treatment is a prophylactic or therapeutic application). Actual methods for preparing administrable compositions will be known or apparent to those skilled in the art and are described in more detail in, for example, Remington's Pharmaceutical Science, 17^{th} Ed., Mack Publishing Co., Easton, Pa. (1985), which is incorporated herein by reference. Preferably the formulation contains from about 50 to about 100 mgs tetracycline, preferably doxycycline, per ounce of total liquid in the formulation. The formulation is prepared by mixing the tetracycline with the liquid vehicle and shaking the mixture.

Alternative forms of the therapeutic composition of the present invention include lozenges, chewing gum, toothpaste, salve, ointments and gels. The compositions also can be provided in the form of a kit, including the tetracycline and the liquid vehicle, optionally together with instructions for use in treating the particular malady or maladies of concern. The instructions preferably include suitable dosage amounts for chronic and/or acute treatment, as well as frequency of administration guidelines.

In the preferred form of the present invention, the therapeutic composition is a mouthwash or a rinse, nose drops, ear drops, or a lozenge, and is self-administered. For example, the mouthwash or rinse can be introduced into the mouth and held in the mouth for at least 30 seconds and preferably at least 1 minute. During the holding period, the mouthwash or rinse is preferably swirled around the mouth in order to bathe the teeth and gums in the composition. Gargling with the mouthwash and rinse during this holding period should also be carried out. The mouthwash or rinse is then expelled from the mouth into a waste receptacle. This procedure can be carried out at least once daily, preferably 2-6 times daily. If the mouthwash or rinse is being given for preventative purposes, then the rinse is preferably used twice a day, preferably in the AM and in the PM on a long-term basis and preferably consists of 50 mgs of antibiotic, preferably doxycycline per 1 ounce of liquid vehicle. The rinse can be used continually for many years without diminishing its benefits, and with no adverse side effects. The rinse can be used on an acute basis or for continual periods as long as 3 months, 6 months, 1 year, 2 years, 5 years or greater. Long term use of more than 10 years with no reported adverse side effects is documented. Long term use to maintain general health is particularly preferred. If the mouthwash or rinse is being used to treat an acute infection, the dosage of antibiotic, preferably doxycycline, is doubled to 100 mgs per ounce of liquid vehicle and the rinse is preferably used between 4 to 6 times a day during the period of acute infection. Nosedrops are particularly useful for treating or preventing sinusitis. If nosedrops comprising the therapeutic composition of the invention, are used, between 3 to 10, preferably 4 to 7, and more preferably 5 drops should be placed in each nostril between 3 to 5 times per day, based on the severity of the infection. This should be done for approximately one week. Administration of the composition of the present invention to the oral cavity cleanses the oral cavity of microorganisms and/or viruses, and reduces or eliminates inflammation and concomitant diseases and conditions associated therewith. Additionally, reduction or elimination of microorganisms and/or viruses in the oral cavity reduces the concentration of bacteria and/or viruses that are absorbed into the bloodstream, and therefore reduces their effect throughout the body.

Without intending to be bound to any mechanism of action, it is believed that cleansing the oral cavity with a therapeutic composition of the invention prevents the blood from being contaminated and therefore prevents and aids in treating diseases such as heart disease, cancer and ulcers caused in part or in whole by toxins or contaminants which have entered the bloodstream through the oral cavity and have attached themselves to the walls of small blood vessels in the heart, pancreas, breast, stomach, bowel, etc. and serve as irritants to cause ulcers, intestinal irritations, and growths and plaque in blood vessels.

The terms “treatment” or “treating” or “treat(s)” as used herein in reference to diseases include 1) preventing such diseases from occurring in a subject who may be predisposed to such disease or diseases but who has not yet been diagnosed as having it or them; 2) inhibiting these diseases, i.e. arresting their development; or 3) ameliorating or relieving the symptoms of these diseases, i.e., causing regression of the disease state. “Therapeutic” as used herein includes compositions for prophylactic treatment of diseases.

The amount of antibiotic, e.g. doxycycline, used in the therapeutic compositions and the dosage selection is an amount and dosage effective to achieve anti-infective and/or triglyceride lowering activity, i.e. the amount necessary to effectively treat the infection or elevated levels of triglycerides, total cholesterol or LDL. Dosage regimens will depend on the dosage and effectiveness thereof, the intended use, the route of administration, the severity of the disease, the body weight of the mammal or human being treated and the patient's general state of health. Although suitable amounts of antibiotic in the therapeutic composition are within the range of 50 to 100 mg per ounce of liquid vehicle, those skilled in the art will determine optimum concentrations and dosages from clinical experience in order to carry out the method of the invention. For example, the physician or dentist treating the patient may begin the patient on a normal strength mouth rinse of 50 mg per ounce of liquid vehicle and then increase the strength of the dose and potentially add nose drops where indicated. Blood cultures and growth cultures can be done after the rinse is used in order to define the optimum dosage and effectiveness more precisely. The amount of therapeutic composition used to treat the patient is defined herein as a therapeutically effective dose.

### EXAMPLE 1

A 100 mg and a 50 mg capsule of doxycycline were opened and the contents added to 3 ounces of antiseptic mouthwash. This mixture was shaken until the doxycycline was dispersed in the mouthwash.

A patient with gum infection administered one third ounce of the mixture into the mouth and swirled in the mouth for 30 seconds and gargled, three times daily for three months. The upper molar gum infection healed within several days. Gumline recession ceased. Stools normalized and sinuses cleared, despite a history of chronic sinusitis. Taste bud sensitivity was enhanced.

### EXAMPLE 2

Two 100 mg capsules of doxycycline were opened and the contents added to 3 ounces of the preferred rinse described above. This mixture was shaken until the doxycycline was dispersed in the rinse.

A patient with elevated total cholesterol (246 mg/dl), elevated triglycerides (379 mg/dl), elevated LDL (139 mg/dl) and low HDL (31 mg/dl) in 1994 administered one third ounce of the mixture into the mouth and swirled in the mouth and gargled for about 30 seconds, three to four times daily for about eight years. The patient was taking no other medication. Blood tests indicated that total cholesterol was lowered to 178, triglycerides to 110, LDL to 99 and HDL raised to 46.

### EXAMPLE 3

Two 100 mg capsules of doxycycline were opened and the contents added to 3 ounces of the preferred rinse described above. This mixture was shaken until the doxycycline was dispersed in the rinse.

A patient with elevated total cholesterol (224 mg/dl), triglycerides (68 mg/dl), LDL (123 mg/dl) and HDL (68 mg/dl) in 1992 administered one third ounce of the mixture into the mouth and swirled in the mouth and gargled for about 30 seconds, three to four times daily for about ten years. The patient was taking no other medication. Blood tests indicated that total cholesterol was lowered to 180, triglycerides to 39, LDL to 103 and HDL raised to 107.

What is claimed is:

1. A therapeutic composition having triglyceride lowering activity comprising a triglyceride-lowering effective amount of a tetracycline, said therapeutic composition being in oral delivery form and adapted for self-administration.

2. The therapeutic composition of claim 1, wherein said tetracycline has not been chemically modified to eliminate antimicrobial efficacy.

3. The therapeutic composition of claim 1, wherein said therapeutic composition is devoid of a morpholinoamino alcohol or pharmaceutically acceptable salt thereof

4. The composition of claim 1, further comprising a liquid vehicle.

5. The composition of claim 4, wherein said liquid vehicle comprises at least 26% alcohol by volume.

6. The composition of claim 4, wherein said liquid vehicle further comprises a mixture of two or more of thymol, menthol, eucalyptol and methylsalicylate.

7. The composition of claim 1 in the form of a mouthwash.

8. The composition of claim 1, wherein said tetracycline is doxycycline.

9. A method of treating or preventing elevated triglyceride levels in a patient comprising cleansing the oral cavity of said patient with the therapeutic composition of claim 1.

10. The method of claim 9, wherein said mammal is a human.

11. The method of claim 9, wherein said cleansing is self-administered.

12. The method of claim 9, wherein said composition is an oral rinse.

13. A method of treating or preventing elevated total cholesterol levels in a patient comprising cleansing the oral cavity of said patient with the therapeutic composition of claim 1.

14. A method of treating a mammal suffering from periodontal disease, comprising administering to said mammal an effective amount of a therapeutic composition comprising a tetracycline which has not been chemically modified to eliminate antimicrobial efficacy, with the proviso that said therapeutic composition is devoid of amorpholinoamino alcohol or pharmaceutically acceptable salt thereof.

15. The method of claim 14, wherein said periodontal disease is gingivitis.

16. The method of claim 14, wherein said tetracycline is doxycycline.

17. A therapeutic composition for preventing the common cold or pneumonia, comprising an effective amount of a tetracycline, said therapeutic composition being in oral delivery form and adapted for self-administration.

18. A kit, comprising a tetracycline, a liquid vehicle for said tetracycline, and instructions for use of said tetracycline and liquid vehicle for treating a malady selected from the group consisting of elevated blood levels of triglycerides, total cholesterol, the common cold and pneumonia.

19. The kit of claim 18, wherein said tetracycline is doxycycline.

20. A method of treating a mammal suffering from a bacterial infection, comprising administering to said mammal an effective amount of a therapeutic composition comprising an antibiotic and a tetracycline, said therapeutic composition being in oral delivery form and adapted for self-administration.
